(19) United States
(12) Patent Application Publication
(10) Pub. No.: US 2006/0286185 A1
(43) Pub. Date: Dec. 21, 2006

(54) Veterinary composition and method of using same

(76) Inventor: Walter George Prokosch, Crosby, TX (US)

Correspondence Address: Walter G. Prokosch, 2142 White Feather Tr., Grosby, TX, 77532, US

(21) Appl. No.: 11/154,345

(22) Filed: Jun. 16, 2005

(51) Int. Cl. A61K 36/899; A61K 36/13; A61K 31/125; A61K 31/20

(52) U.S. Cl. 424/750; 514/692; 514/558; 424/770

(57) ABSTRACT

This invention relates to a topical veterinary composition and method of using for the treatment of minor flesh wounds or lacerations in animal and to promote the healing thereof The composition comprises of the active ingredients of tall oil, wheat germ oil and myristic acid either per se or as a topical veterinary ointment comprising the active ingredient in admixture with a suitable carrier and/or antiseptic.

### BACKGROUND OF THE INVENTION

(1) Field of Invention

This invention relates to a veterinary composition and method of using same. More particularly the invention concerns a topical veterinary composition for the treatment of skin wounds, lacerations, and abrasions in animals, to promote the healing thereof, and comprising as the active ingredients of a variety of fatty acids (derived from the tall oil fatty acid group}, myristic acid, and wheat germ oil containing vitamin E. In another aspect the invention concerns a method for treating flesh wounds, or lacerations in animals to promote the healing thereto either per se or as a topical veterinary ointment comprising the active ingredient in admixture with a suitable carrier and/or antiseptic.

(2) Description of the Prior Art

Veterinary agents for the treatment of flesh wounds, lacerations, and skin abrasions in animals to promote the healing thereof are known. One such agent is a topical wound spray Derma G*O, covered by [U.S. Pat. No. 4,443,437 dated Apr. 17, 1984] comprising of an alcoholic solution of tall oil and camphor. Another known wound treating agent comprises furazolidone as the active ingredient.

Whilst such agents area effective in many cases, they have been found to be less effective in certain cases of severe skin wounds. A need therefore exists for an improved agent for promoting the healing of skin wounds in animals.

### SUMMARY OF INVENTION

In accordance with the present invention it has been found that a combination of certain fatty acids such as palmitic, oleic, linoleic, palmitoleic, stearic, pimaric, abietic acids, which are the major components of tall oil fatty acids coupled with addition of myristic acid and wheat germ oil, containing vitamin E, is an improved healing agent.

### DETAILED DESCRIPTION

The invention is based on the discovery that the combination of wheat germ oil which is high in vitamin E and myristic acid in conjunction with tall oil fatty acids is an improved therapeutic agent in the treatment of flesh wounds, lacerations, and fistulas in animals.

Wheat Germ Oil is a product of cold pressed wheat germ from the inner body of granular wheat.

Myristic Acid is a natural occurring fatty acid in nutmeg and also in butter fat. It is currently available as a synthetically produced product from several suppliers, including ACME Hardesty Inc.

Tall oil fatty acid is a by-product of the paper-making industry and is obtained by acidifying the black liquor skimming, resulting from the alkaline treatment of wood pulp, particularly in the sulfate (Kraft) process of wood pulp manufacture.

Crude tall oil is essentially a mixture of rosin (e.g. abietic and pimaric) acids and fatty (primary oleic and linoleic, but also compromising small amounts of others such as palmitic, palmitoleic, stearic, and other C₁₇-C₂₂ acids in isomeric or conjugated form) acids in approximately equal proportions, plus a small proportion (e.g. up to about 10% by weight) of neutral material comprising primarily esters of fatty acids but containing small amounts of sterols, higher alcohols and hydrocarbons. The precise composition of tall oil fatty acids will vary with the original wood source used to prepare the wood pulp and with the degree of refinement of the tall oil after acidification; thus, the term tall oil fatty acids used herein includes the acidic mixture obtained by the acidification of the black liquor resulting from the alkaline pulping of wood pulp from any source, and includes refined (i.e. distilled) tall oil fatty acids, as well as the crude product Suitable tall oil products for use in the present invention are available from MeadWestvaco, under trade names such as Meadwestvaco M-15 or M-28B, and from Arizona Chemical Co. under the trademark ACTINOL FA.

The tall oil fatty acids may be applied as part of the composition of a topical medicament directly to the wounded area of the animal as often as may be desired to promote healing, but usually one to three times a day for several days or until the wound is healed. More usually, however, for use in accordance with this invention, the tall oil fatty acids will be formulated as a topically administrable composition (i.e. as an ointment, lotion, spray, or aerosol), comprising the active ingredient in admixture with a suitable topically administrable carrier or diluent.

Particularly suitable carriers or diluents which may be used in the compositions of this invention are low molecular weight alcohols, especially isopropyl alcohol also known as isopropanol. Other suitable carriers or diluents will be apparent to those skilled in the art. The proportions of tall oil fatty acids to the carrier are in no way critical.

In addition to the active tall oil fatty acid ingredient, the compositions of this invention may contain minor proportions by weight of other active ingredients and, particularly, an antiseptic, such as camphor, or other veterinary biologically acceptable antiseptic compounds.

A particularly preferred, but exemplary, liniment solution in accordance with this invention comprises:

| | |
|---|---|
| Tall Oil Fatty Acids | 20-70% |
| Isopropyl Alcohol | 30-60% |
| Camphor | 5-15% |
| Wheat Germ Oil | 1-5% |
| Myristic Acid | 1-5% |

The invention is illustrated by the following Examples.

### EXAMPLE 1

A dog (dachshund) which had been injured in a fight with a large dog. The injury consisted of approximately 50 square inches of skin being torn loose from an area just behind the collar on his neck to the middle of his back. This area was sutured back to his healthy skin at the veterinary clinic, but would not heal back. After the skin started slough off, the veterinary had to remove the remaining dead skin. At this point treatment was started by applying a spray, twice a day of the topical liniment composed of:

| | |
|---|---|
| Tall Oil Fatty Acids | 20-70% |
| Isopropyl Alcohol | 30-60% |
| Camphor | 5-15% |
| Wheat Germ Oil | 1-5% |
| Myristic Acid | 1-5% |

After seven days of treatment the start of healing was noted and after six additional weeks 95% of the denuded area had been covered over with new skin.

### EXAMPLE 2

A colt with lacerated front leg denuded of skin tissue sufficient to expose the tendons and, in one area approximately 2 cm ×8 cms of the third metatarsal was presented for treatment. Initial treatment over 5 days using furazolidone ointment showed no healing. Treatment was then commenced with a liniment substantially (as described in Example 1) applied liberally as a spray twice daily to the wounded area. After two days, healing was already noticeable and at 2 weeks the wound was found to be granulated in. Six weeks later it had fully healed and with hair growth.

### REFERENCES

Publications: Cannon, Talloils; Chem. Engineering, June 1954, pp. 142-146

What we claim is:

1. A method of treating wounds, laceration or surface fistulas in animals to promote the healing thereof, which comprises topically applying a effective amount tall oil fatty acids, in combination with wheat germ oil and myristic acid to the affected area for promoting healing thereof.

2. A method according to claim 1, in which the affected areas is treated with a liniment comprising, on a weight basis:

| | |
|---|---|
| Tall Oil Fatty Acids | 20-70% |
| Isopropyl Alcohol | 30-60% |
| Camphor | 5-15% |
| Wheat Germ Oil | 1-5% |
| Myristic Acid | 1-5% |
